(19) United States
(12) Patent Application Publication
(10) Pub. No.: US 2026/0007619 A1
(43) Pub. Date: Jan. 8, 2026

(54) Sublingual Epinephrine Compositions Including pH-Modifying Excipients And Penetration Enhancers And Methods for Use Thereof

(71) Applicant: Nova Southeastern University, Fort Lauderdale, FL (US)

(72) Inventor: Mutasem Rawas-Qalaji, Fort Lauderdale, FL (US)

(73) Assignee: Nova Southeastern University, Fort Lauderdale, FL (US)

(21) Appl. No.: 19/270,386

(22) Filed: Jul. 15, 2025

Related U.S. Application Data
(63) Continuation of application No. 17/285,711, filed on Apr. 15, 2021, now Pat. No. 12,357,591.
(60) Provisional application No. 62/748,117, filed on Oct. 19, 2018.

(51) Int. Cl. A61K 31/137; A61K 9/00; A61K 47/12; A61K 47/20

(52) U.S. Cl. CPC A61K 31/137; A61K 9/0056; A61K 9/006; A61K 47/12; A61K 47/20

(57) ABSTRACT

The invention provides sublingual epinephrine compositions including epinephrine fine particles formulated with pH-modifying excipients and penetration enhancers. The sublingual compositions are used to control absorption of epinephrine at the site of delivery in an oral cavity. The invention also provides methods for therapeutic use of the sublingual compositions for treatment of conditions responsive to epinephrine and/or for increasing sublingual bioavailability of epinephrine.

### FIELD OF THE INVENTION

The invention is encompassed within the field of drug formulation and generally relates to modifications in drug formulation that result in improved delivery of sublingually-administered medication, particularly to modifications in drug formulation that enhance permeability of the active ingredient of the sublingually-administered medication, and most particularly to sublingual epinephrine compositions including epinephrine fine particles formulated with pH-modifying excipients and penetration enhancers that improve sublingual delivery/absorption of epinephrine.

### BACKGROUND

Epinephrine (Epi) is the drug of choice for the emergency treatment of anaphylaxis (1-4). For out-of-hospital emergency treatment of anaphylaxis, Epi auto-injectors such as EpiPen®, EpiPen Jr® (Mylan Specialty LP, Basking Ridge, NJ, USA) are prescribed. However, self-injectable Epi is underutilized when anaphylaxis occurs (5, 6). The drawbacks of Epi auto-injectors include: high cost which limits affordability and availability worldwide (6), bulkiness, limitations if repeat dosing is required (7), fear and anxiety associated with the use of needles, and dosing errors due to incorrect administration technique (8).

Epinephrine is extensively metabolized after oral administration by the catechol-O-methyltransferase in the gastrointestinal tract and by monoamine oxidase in the gastrointestinal tract and in the liver (9). In aqueous solutions, Epi is unstable in the presence of light, oxygen, heat, and neutral or alkaline pH values. Epi decomposes into neurotoxin molecules, adrenochrome, and adrenolutin (10).

The sublingual route of administration is a promising alternative route for Epi administration. Drugs that can be absorbed sublingually bypass potential metabolic conversion in the gastrointestinal tract and hepatic first-pass metabolism and reach the systemic circulation in a pharmacologically-active form (11-14). The high vascularity of the sublingual mucosa and the low molecular weight of Epi facilitate its rapid absorption directly into the venous circulation.

Orally-disintegrating tablets, which disintegrate rapidly in the minimal volume of the oral saliva, are good candidate for the sublingual administration of Epi (15, 16). They would release Epi instantly into the sublingual cavity to be rapidly absorbed through the sublingual mucosa into the systemic circulation (17).

The optimal pH values for the stability of Epi in aqueous solution are 3.0-3.8 (10). However, the pH of the saliva under normal conditions varies between 5.8 to 7.1. Also, some pharmaceutical excipients and acidic or alkaline drinks may alter the normal saliva pH, which may affect the stability of sublingually administered Epi (18).

The administered sublingual tablets are recommended to be kept under the patient's tongue until it is completely disintegrated or dissolved and can no longer be felt under the tongue. The time for the complete tablet disintegration or dissolution under the patient's tongue should not be more than 2 to 5 minutes and patients are instructed not to eat or drink anything during this time. The required therapeutic dose of the administered drug should be already absorbed during this time into the systemic circulation through the sublingual mucosa. Although the contact time between Epi and saliva in the sublingual cavity will be for a short period of time, there is no evidence in the literature that indicates Epi will be stable during its sublingual administration and how fluctuations in the saliva pH may affect its stability. Therefore, it is very crucial to evaluate how the change in the saliva pH due to various drinks and potential pharmaceutical excipients used in the tablet formulation would affect the stability of Epi during its sublingual administration. Furthermore, the stability of Epi in human saliva during its sublingual administration needs to be evaluated for any potential enzymatic degradation of Epi that may take place during its sublingual administration and to assess the feasibility of using the sublingual route as a potential route for the administration of Epi for the first-aid treatment of anaphylaxis.

Also, the permeability of drugs depends on their degree of ionization at the site of administration (19). The degree of ionization of weak basic or acidic drugs is based on prevailing pH. pH-modifying excipients incorporated into the dosage form can alter absorption medium's, therefor, can affect the permeability and bioavailability of administered drugs. The selection of excipients to be used in a dosage form depends on the properties of the target absorption site. The addition of pH modifiers into a tablet formulation designed for sublingual administration ensures that the pH of the saliva at the absorption site is controlled within the range that is optimal for drug absorption and reduces absorption's variabilities due to individual saliva pH variability, or due to any potential effect of food and drinks on saliva pH. The selection of these excipients is particularly important for Epi as a weak base. Its extent of absorption can be greatly dependent on its degree of ionization, which is mainly affected by the pH of the saliva. Therefore, the evaluation of various pH-modifying excipients is critical for enhancing Epi sublingual permeability and absorption, therefore, its bioavailability.

Another approach to enhance drug absorption is use of penetration (permeability) enhancers. There is a wide range of enhancers that have been evaluated at various concentrations for their ability to enhance drug absorption and for their safety (20). The mechanism of permeation enhancement can be utilized for hydrophilic and lipophilic drugs through paracellular diffusion, transcellular diffusion, or both. Epi has log P of −1.37 (21), which can be a candidate for using a penetration enhancer to increase it absorption. Previous in vivo studies have shown that the relative sublingual bioavailability of Epi is low when compared to the intramuscular injection of Epi (22). Therefore, the assessment of various penetration enhancers to be incorporated into the dosage form to be administered sublingually on Epi sublingual permeability is needed. A wide range of permeation enhancers can be used at various concentrations to enhance Epi permeability. For example, sodium dodecyl sulfate (SDS) can be used at concentration that ranges from 0.05% to 1% (23, 24) and palmitoyl carnitine chloride (PCC) can be used at concentration that ranges from 0.05 mM to 0.35 mM (25, 26) with minimal and revisable local tissue damage (27).

Without being bound by theory, it is thought that fabrication of epinephrine into fine particles, including epinephrine nanoparticles or nanocrystals and epinephrine microparticles or microcrystals, and incorporation of the epinephrine fine particles into a tablet formulation with pH-modifying excipients and penetration enhancers will significantly increase the permeability of epinephrine and thus will significantly increase the absorption of the sublingually-administered epinephrine resulting with reduction of sublingual epinephrine dose required for both adults and children.

Epinephrine is an important medication in health systems worldwide for management of life-threatening allergies, i.e. anaphylaxis, cardiac events, i.e. cardiac arrest, and breathing difficulties, i.e. asthma, bronchial asthma, bronchitis, emphysema, and respiratory infections. It would be very advantageous to have non-invasive sublingual drug delivery of epinephrine as a potential alternative, patient-friendly, convenient, and cost-effective dosage form.

### SUMMARY OF THE INVENTION

Epinephrine (Epi) is life-saving in the treatment of anaphylaxis. In community settings, a first-aid dose of epinephrine in an amount of 0.1 mg, 0.15 mg, or 0.3 mg is injected into the mid-outer thigh by patients or caregivers using an auto-injector such as an EpiPen® (epinephrine auto-injector 0.3 or 0.15 mg, Mylan Inc., Basking Ridge, NJ) or Auvi-Q® (epinephrine auto-injector 0.1, 0.3, or 0.15 mg, kaleo, Inc., Richmond, VA). Epi auto-injectors are under-used because of needle phobia, bulky size, and high cost; additionally, there are only two to three fixed doses, shelf-life is only 12-18 months, and unintentional injection and injury sometimes occur.

The instant invention circumvents the aforementioned problems by providing a fast-disintegrating epinephrine tablet formulation for anaphylaxis treatment. Although this formulation was designed with regard to anaphylaxis, it is equally effective and contemplated for use in treatment of any condition responsive to epinephrine such as cardiac events, i.e. cardiac arrest, and breathing difficulties, i.e. asthma, bronchial asthma, bronchitis, emphysema, and respiratory infections.

In a validated rabbit model, this fast-disintegrating epinephrine tablet formulation resulted in plasma epinephrine concentrations similar to those achieved after a 0.3 mg epinephrine intra-muscular injection. (22) Furthermore, epinephrine was stable in these fast-disintegrating tablets for at least seven years.

The instant inventor hypothesized that adjusting the pH of the sublingual microenvironment that surrounds Epi tablet using alkalizing excipient can modify the pH of the saliva in that area (tablet's diffusional layer or the stagnant unstirred layer that forms around the tablet and contains the dissolved Epi from the tablet) and reduce Epi ionization without affecting its stability, which will increase Epi permeability. Also, incorporating a penetration enhancer in the sublingual tablet formulation can increase Epi permeability as well. Some experiments, which led to the inventive compositions, are described herein: 1) evaluate the stability of Epi in various media, 2) evaluate the effect of modifying the pH of the diffusion medium on enhancing Epi sublingual permeability, 3) evaluate the ability of pH-modifying excipients to alter the microenvironment's pH of the absorption medium of Epi tablet, and 4) evaluate the effect of using various penetration enhancers on enhancing Epi sublingual permeability.

One of the most common approaches to enhance the rate of drug dissolution and absorption is to significantly reduce its particle size to the micro- or nano-size range. Drug nanocrystals (NC) or microcrystals (MC) are advantageous due to the minimal required excipients and almost 100% of the pure drug is produced during the fabrication process (28). Also, the collected dried drug NC or MC can be formulated into various dosage forms.

The phrase “epinephrine fine particles” refers to epinephrine particles of about 2.5 μm or less to about 500 nm-100 nm in size and includes epinephrine nanoparticles or nanocrystals and epinephrine microparticles or microcrystals.

In one aspect, the invention provides epinephrine fine particles.

In one aspect, the invention provides epinephrine nanoparticles. The epinephrine can be either an epinephrine base epinephrine bitartrate salt or other effective epinephrine salt, or prodrug of epinephrine.

In another aspect, the invention provides epinephrine nanocrystals. A nanocrystal is a nanoparticle having a crystalline structure. The term “nanocrystal” is a more specific term for describing a nanoparticle. A drug nanocrystal contains almost 100% pure drug, thus an epinephrine nanocrystal contains almost 100% pure epinephrine. A drug nanoparticle can include nanocrystals or a drug encapsulated within a polymer at different ratios. One example is the epinephrine nanoparticles comprising chitosan and tripolyphosphate (TPP) described in the previously-filed related application; U.S. Provisional Patent Application Ser. No. 61/550,359, filed on Oct. 21, 2011.

The term “about” as used in this application refers to a quantity at or near a defined quantity which will still allow for desired function.

In a further aspect, the invention provides improvements in delivery of sublingually-administered medication, particularly, but not limited to, enhancing permeability of the active ingredient of the sublingually-administered medication.

In an embodiment, the invention provides sublingual epinephrine compositions including epinephrine fine particles formulated with pH-modifying excipients and penetration enhancers.

In one aspect of this embodiment, the invention encompasses a sublingual composition formulated as a fast-disintegrating tablet. This composition includes epinephrine fine particles, for example, but not limited to epinephrine bitartrate fine particles, and one or both of a pH-modifying excipient and a penetration enhancer. A preferred, non-limiting epinephrine is epinephrine bitartrate equivalent to about 20 mg or less epinephrine, 15 mg or less epinephrine, or preferably ranging from about 20 mg to about 1 mg epinephrine. Non-limiting examples of a pH-modifying excipients are the alkalizing agents, sodium carbonate, sodium bicarbonate, and calcium citrate. These alkalizing agents can be added in any effective percentage to the inventive compositions, for example, but not limited to sodium bicarbonate added at a percentage of 0.75% w/v or 10% w/w of tablet weight. Non-limiting examples of penetration enhancers are sodium dodecyl sulfate (SDS) or palmitoyl carnitine chloride (PCC). These penetration enhancers can be added in any effective percentage to the inventive compositions, for example, but not limited to SDS added at 0.075% w/v or 1% w/w of API used and PCC added at 1.2% w/v or 16% w/w of API used.

In yet another aspect, the invention provides methods for controlling the microenvironment at the site of drug delivery in an oral cavity. For example, the invention provides a method for controlling pH of saliva within a range optimal, preferred, but not limited to a range of about 6.8 and about 8, for absorption at a site of sublingual administration in a subject in need thereof. In another similar aspect, the invention provides a method for altering ionization of epinephrine at a site of sublingual administration of the epinephrine in a subject in need thereof. Both methods include providing a composition including epinephrine fine particles and at least one of a pH-modifying excipient and a penetration enhancer; and administering the composition to the subject. Control of the pH of saliva preferably includes increasing the pH.

In another aspect, the invention provides a composition including epinephrine nanoparticles or nanocrystals capable of enhancing the sublingual bioavailability of epinephrine for the emergency treatment of anaphylaxis and/or other conditions responsive to epinephrine.

In another aspect, the invention provides “oral disintegrating tablets (ODTs)” including epinephrine nanoparticles or nanocrystals or epinephrine microparticles or microcrystals.

As described herein, buccal or sublingual oral disintegrating tablets (ODTs) are distinguished from conventional sublingual tablets, lozenges, or buccal tablets by the ODTs' ability to fully dissolve or disintegrate in less than about one minute in the mouth.

The invention also provides pharmaceutical compositions including epinephrine nanoparticles or nanocrystals or epinephrine microparticles or microcrystals in ODT form.

The invention also provides a pharmaceutical composition including epinephrine nanoparticles or nanocrystals or epinephrine microparticles or microcrystals and a pharmaceutically-acceptable carrier for buccal or sublingual administration.

The phrase “pharmaceutically-acceptable carrier” refers to an inactive and non-toxic substance used in association with an active substance, i.e. epinephrine, especially for aiding in the application of the active substance. Non-limiting examples of pharmaceutically-acceptable carriers are diluents, binders, disintegrants, flavorings, fillers, and lubricants. Pharmaceutically-acceptable carriers can have more than one function, i.e. a filler can also be a disintegrant. Additionally, pharmaceutically-acceptable carriers may also be referred to as non-medicinal ingredients (NMIs).

The invention also provides a pharmaceutical composition, for buccal or sublingual administration, including epinephrine nanoparticles or nanocrystals or epinephrine microparticles or microcrystals and at least one of a pharmaceutically-acceptable carrier, a surfactant, a penetration enhancer, a pH-modifying excipient, and a mucoadhesive. The pharmaceutical composition can further include at least one of a taste enhancer and a sweetening agent and mouthfeel enhancer. A non-limiting example of a taste enhancer is citric acid. Citric acid masks the bitter taste of epinephrine. A non-limiting example of a sweetening agent and mouthfeel enhancer is mannitol. The pharmaceutical composition can further include at least one of a filler, a lubricant, and a disintegrant. Non-limiting examples include microcrystalline cellulose (filler), magnesium stearate (lubricant), and low-hydroxypropyl ethers of cellulose (disintegrant).

Additionally, the invention provides a pharmaceutical composition including epinephrine nanoparticles or nanocrystals or epinephrine microparticles or microcrystals, in which the bitter taste of the epinephrine is masked by a taste enhancer. A non-limiting example of a taste enhancer is citric acid.

In another aspect, the invention provides a method for enhancing sublingual bioavailability of epinephrine in a subject in need thereof including steps for providing a composition including epinephrine nanoparticles or nanocrystals or epinephrine microparticles or microcrystals and at least one pharmaceutically-acceptable carrier and administering the composition to the subject. The described fast-disintegrating epinephrine tablets enhance bioavailability of epinephrine by releasing epinephrine within sixty seconds of administration. The term “subject” includes any human being or animal that can benefit from the inventive compositions and methods. The term “patient” is also used herein to refer to the subject.

In another aspect, the invention provides a method for treating a condition responsive to epinephrine in a subject in need thereof including steps for providing a composition including epinephrine nanoparticles or nanocrystals or epinephrine microparticles or microcrystals and at least one pharmaceutically-acceptable carrier and administering the composition to the subject. Conditions responsive to epinephrine react to administration of epinephrine. Non-limiting examples of conditions responsive to epinephrine include a cardiac event, i.e. cardiac arrest, or an allergic reaction. i.e. anaphylaxis, asthma, or bronchial asthma.

The phrase “effective amount” refers to the amount of a composition necessary to achieve the composition's intended function.

The phrase “pharmaceutically-effective dose” refers to the amount of a composition necessary to achieve a desired pharmaceutical effect. It is often desirable to use the smallest effective dose of a drug. One example of a dose range for the described epinephrine nanoparticles or nanocrystals or epinephrine microparticles or microcrystals is approximately 1 mg to 40 mg epinephrine nanoparticles or nanocrystals or epinephrine microparticles or microcrystals.

The phase “therapeutically-effective amount” refers to the amount of a composition required to achieve the desired function, i.e. treatment of the condition responsive to epinephrine.

In another aspect, the invention provides a method for treating a breathing difficulty in a subject in need thereof including steps for providing a composition including epinephrine nanoparticles or nanocrystals or epinephrine microparticles or microcrystals and at least one pharmaceutically-acceptable carrier and administering the composition to the subject. Breathing difficulties responsive to epinephrine include, but are not limited to, breathing difficulties associated with anaphylaxis, asthma, bronchial asthma, bronchitis, emphysema, and respiratory infections.

The invention additionally provides a method for treatment of an allergic emergency in a subject diagnosed with or suspected of having an allergic emergency including steps for providing a composition including epinephrine nanoparticles or nanocrystals or epinephrine microparticles or microcrystals and at least one pharmaceutically-acceptable carrier and administering the composition to the subject. Non-limiting examples of allergic emergencies are anaphylaxis, asthma, and bronchial asthma.

In an additional aspect, the invention provides a method for treatment of a cardiac event in a subject diagnosed with or suspected of having a cardiac event including steps for providing a composition including epinephrine nanoparticles or nanocrystals or epinephrine microparticles or microcrystals and at least one pharmaceutically-acceptable carrier and administering the composition to the subject. A non-limiting example of a cardiac event is cardiac arrest.

Any of the above-described epinephrine fine particles (including epinephrine nanoparticles or nanocrystals and epinephrine microparticles or microcrystals), compositions, and pharmaceutical compositions can be formulated for buccal or sublingual administration, particularly those epinephrine fine particles (including epinephrine nanoparticles or nanocrystals and epinephrine microparticles or microcrystals), compositions, and pharmaceutical compositions intended for use in emergency situations.

In another aspect, any of the above-described epinephrine fine particles (including epinephrine nanoparticles or nanocrystals and epinephrine microparticles or microcrystals) can be used in the manufacture of any of the above-described compositions and pharmaceutical compositions.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 is a graph illustrating epinephrine recovery (%) in various pH level, Mean±SD (n=3).

FIG. 2 is a graph illustrating epinephrine recovery (%) in human saliva, Mean±SD (n=3).

FIG. 3 is a graph illustrating cumulative permeated epinephrine and percentage through porcine sublingual mucosa over time at various pH, Mean±SD (n=4).

FIG. 4 is a graph illustrating AUC₀₋₉₀ of permeated epinephrine through porcine sublingual mucosa at various pH, Mean±SD (n=4).

FIG. 5 is a graph illustrating epinephrine influx and permeability through porcine sublingual mucosa at various pH, Mean±SD (n=4).

FIG. 6 is a graph illustrating cumulative permeated epinephrine and percentage through porcine sublingual mucosa over time with various permeation/penetration enhancers, Mean±SD (n=4).

FIG. 7 is a graph illustrating cumulative permeated epinephrine and percentage through porcine sublingual mucosa over time with various formulations, Mean±SD (n=4).

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described compositions, formulations, epinephrine fine particles, methods, procedures, and techniques described herein along with any further application of the principles of the invention as described herein, are contemplated as would normally occur to one skilled in the art to which the invention relates.

As noted above, the experiments described herein had defined objectives leading to development of the inventive compositions.

First, the short-term stability of epinephrine (Epi) at wide range of pH was assessed. Modification of the pH of the absorption's microenvironment can alter the degree of drug ionization and therefore its permeability. Altering the saliva pH (5.5-7.2) in order to enhance Epi sublingual absorption and reduce its absorption variability can, on the other hand, negatively affect its stability. It was thought that altering Epi ionization by modifying the pH of absorption medium, without negatively affecting Epi stability, can enhance Epi permeability and optimize sublingual Epi delivery as an alternative route for the treatment for anaphylaxis and other conditions responsive to Epi.

Epi was found to be relatively stable for the time-period required for its sublingual administration at the saliva pH and up to pH 8. Altering the pH of the absorption's microenvironment up to pH 8 to enhance Epi sublingual delivery and reduce its absorption variability should not affect its stability.

Next, the effect of modifying the pH on the sublingual permeability of epinephrine (Epi) in order to alter its degree of ionization was evaluated. It was thought that altering Epi ionization by modifying the pH of absorption medium can enhance Epi permeability and optimize sublingual Epi delivery as an alternative route for the treatment for anaphylaxis and other conditions responsive to Epi.

It was found that increasing the pH of the diffusion medium and the subsequent reduction in Epi ionization can result in a significant increase in the Epi sublingual permeation.

Finally, the effect of different permeability enhancers on the sublingual permeability of epinephrine (Epi) was evaluated. It was thought that the incorporation of a permeability enhancer can further enhance Epi sublingual permeability as a potential alternative route of administration for the treatment of anaphylaxis and other conditions responsive to Epi.

It was found that penetration enhancers were able to enhance the sublingual permeability of Epi. Sodium dodecyl sulfate (SDS) was superior to palmitoyl carnitine chloride (PCC) in enhancing the sublingual permeation of Epi. The incorporation of a permeability enhancer into Epi formulation can significantly enhance the permeability and potentially the sublingual absorption of Epi as a potential alternative route of administration for the treatment of anaphylaxis and other conditions responsive to Epi.

### Example 1: Stability of Epinephrine (Epi) in Various Media

Epi optimal stability in aqueous solution is at pH range of 3.0-3.8 (10). However, the normal saliva pH is between 5.8-7.1, which can be also altered due to food, drinks, or excipients used in the sublingual dosage form. pH-modifying excipients can be used in the sublingual formulation to alter the degree of drug ionization and therefore its permeability. However, modifying the saliva pH using pH-modifying excipients in order to enhance Epi sublingual absorption and/or reduce its absorption variability due to food or drinks can, on the other hand, negatively affect its stability. Therefore, the short-term stability, 20-30 min, of Epi was evaluated in various media to test the feasibility of Epi sublingual administration at various pH values for the treatment for anaphylaxis and other conditions responsive to Epi.

### Stability of Epinephrine in Various pH

Epinephrine bitartrate (EpiBit) equivalent to 20 mg Epi was dissolved in 2 mL of Mcvilian buffer (n=3) (phosphate acetate buffer) at specific pH including pH 1.2, 3.5, 4.5, 5, 5.5, 6, 6.8, 7.4, and 8. Aliquot samples were withdrawn at specific time intervals including 2(maximum expected time for sublingual administration), 4, 6, 10, 15, and 30 min. Withdrawn samples were immediately diluted with a stabilizing solution containing 0.1 M perchloric acid and 0.1 mM sodium metabisulfite to stabilize and stop any potential epi degradation. Collected samples were frozen for further analysis by HPLC for Epi content using the USP method for Epi injection. Epi recovery % at T₀ was used as a positive control for each sample. The mean±SD of Epi recovery (%) in each pH at different time were calculated and statistically compared using repeated measures-ANOVA and Tukey-Kramer Tests (p<0.05).

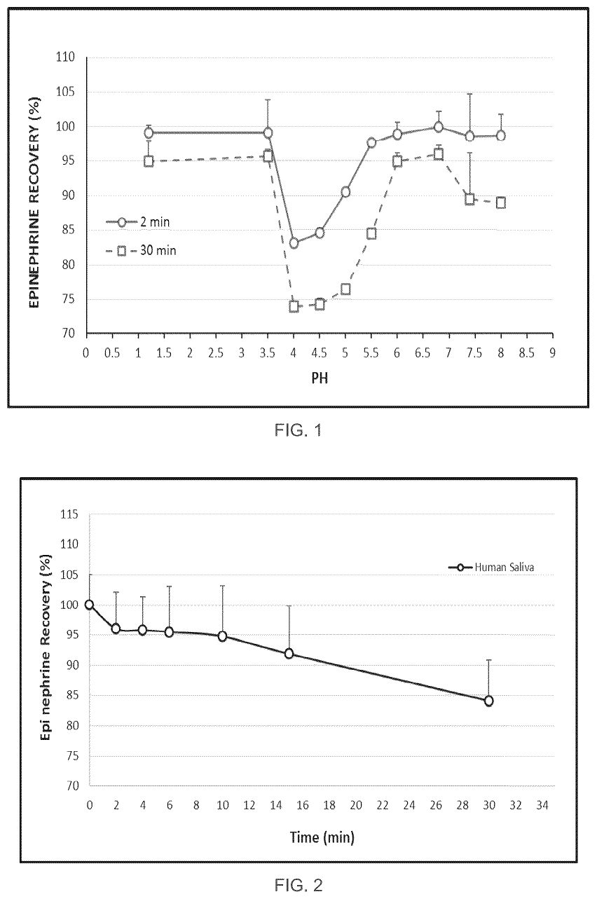

The mean±SD (n=3) Epi recovery (%) at the different time intervals in the various pH tested are shown in Table 1. Mean±SD (n=3) Epi recovery (%) was not significantly different (p>0.05) from the original concentration (positive control T₀) at the different sampling time intervals in pH range from 1.2 to 3.5 and 5.5 to 8 (FIG. 1). At pH 4, 4.5 and 5 Epi recovery (%) declined significantly (p<0.05).

TABLE 1
Mean ± SD (n = 3) epinephrine stability in various pH
Epi Recovery (%)

| Time (min) | pH 1.2 | pH 3.5 | pH 4.0 | pH 4.5 | pH 5 | pH 5.5 | pH 6 | pH 6.8 | pH 7.4 | pH 8 |
|---|---|---|---|---|---|---|---|---|---|---|
| 0 | 100 ± 4 | 100 ± 4 | 100 ± 0 | 100 ± 0 | 100 ± 0 | 100 ± 0 | 100 ± 3 | 100 ± 2 | 100 ± 5 | 100 ± 3 |
| 2 | 99 ± 1 | 99 ± 5 | 83 ± 0.3* | 85 ± 0.7 | 90 ± 0.0* | 98 ± 0.0 | 99 ± 2 | 100 ± 2 | 99 ± 6 | 99 ± 3 |
| 4 | 100 ± 3 | 99 ± 5 | 82 ± 0.2* | 84 ± 0.7* | 90 ± 0.0* | 97 ± 0.0 | 99 ± 2 | 99 ± 2 | 98 ± 6 | 98 ± 3 |
| 6 | 95 ± 4 | 98 ± 5 | 82 ± 0.2* | 82 ± 0.9* | 87 ± 0.0* | 97 ± 0.0 | 98 ± 2 | 98 ± 2 | 96 ± 8 | 96 ± 4 |
| 10 | 96 ± 2 | 96 ± 4 | 80 ± 0.5* | 80 ± 0.7* | 86 ± 0.0* | 96 ± 0.1 | 100 ± 2 | 98 ± 2 | 94 ± 8 | 94 ± 2 |
| 15 | 95 ± 2 | 96 ± 3 | 79 ± 0.6* | 78 ± 0.7* | 84 ± 0.1* | 88 ± 0.1 | 97 ± 3 | 97 ± 2 | 93 ± 8 | 91 ± 3* |
| 30 | 95 ± 3 | 96 ± 1 | 74 ± 0.7* | 74 ± 0.8* | 76 ± 0.0* | 84 ± 0.0* | 95 ± 1 | 96 ± 1 | 89 ± 7 | 89 ± 1* |

*p < 0.05 from T₀

Epi was stable in a wide range of pH values for sufficient time to allow for sublingual drug administration except between pH 4 to 5, which resulted in 10-17% Epi loss in the first 2 min. Epi sublingual administration should be feasible and would have minimal or no degradation due to incorporating pH-modifying excipients into the tablet formulation or due to human consumption of various drinks with wide range of acidity and alkalinity (except between pH 4 to 5) without adjusting the pH.

### Stability of Epinephrine in Human Saliva

Saliva samples were collected from human volunteer. The volunteer was asked not to eat or drink any acidic, alkaline, or spicy food or beverages and to rinse his mouth before the collection of saliva. The saliva was collected into an ice-bathed beaker and then degassed to remove air bubbles and then filtered through 0.45 μm membrane.

EpiBit equivalent to 2 mg Epi was dissolved in 400 μL (equivalent to 10 mg Epi in 2 mL) of saliva (n=3) that was immersed into 37° C. water bath for 30 minutes before the start of the testing study. Aliquot samples were withdrawn at specific time intervals including 2, 4, 6, 10, 15, and 30 min. Withdrawn samples were immediately diluted with Epi stabilizing solution containing 0.1 M perchloric acid and 0.1 mM sodium metabisulfite to stabilize and stop any potential epi degradation. Collected samples were frozen for further analysis by HPLC for Epi content using the USP method for Epi injection. The mean±SD of Epi recovery (%) was calculated.

The mean±SD (n=3) Epi recovery (%) at the different time intervals in human saliva were shown in Table 2 and FIG. 2.

TABLE 2
Mean ± SD (n = 3) epinephrine stability in Human Saliva

| | Epi Recovery (%) |
|---|---|
| Time (min) | Saliva |
| 0 | 100 ± 0.2 |
| 2 | 96 ± 0.2 |
| 4 | 96 ± 0.2 |
| 6 | 95 ± 0.1 |
| 10 | 95 ± 0.2 |
| 15 | 92 ± 0.2* |
| 30 | 84 ± 0.0* |

*p < 0.05 from T₀

Epi was stable in human saliva (saliva pH and up to pH 8) for sufficient time to allow for sublingual drug administration. Epi concentration was only significantly lower than the initial concentration in human saliva after 15 and 30 min (92±0.2% and 84±0.0%, respectively). Epi is suitable for sublingual administration and would have minimal or no significant degradation due to human saliva's pH and composition. Altering the pH of the absorption microenvironment up to pH 8 to enhance Epi sublingual delivery and reduce its absorption variability should not effect stability.

### Example 2: Epinephrine Sublingual Permeability in Various pH

Modification of the pH of the absorption's microenvironment due to food, drinks, or individual saliva variability can cause variability in drug permeability and absorption due to altering its degree of drug ionization. Also, pH-modifying excipients can be incorporated in the sublingual formulation to reduce drug ionization and therefore enhance its permeability and absorption. Therefore, the sublingual permeability of Epi at a wide range of pH was evaluated in order to determine the pH values at which Epi permeability is optimal.

### Excising and Preparing Porcine Sublingual Mucosa

Frozen pig's heads were obtained from local abattoir. Then, they were defrosted at room temperature. The porcine mucosa were excised by dissecting the sublingual mucosa and removing the underlying connective tissue using a scalpel and fine tweezers using established surgical techniques. The excised mucosa were frozen on aluminum foil at −20° C. until used (<4 weeks).

### Epinephrine Permeability Studies

Static vertical jacketed Franz Cells with 01) of 20 mm and reservoir volume 20±1 mL (PermeGear Inc., Hellertown, PA) were used to measure the permeability of Epi at various pH. The sublingual mucosa (floor of the mouth) excised from pigs were used as the diffusion membranes. Mcvilian buffer at various pH was used in the donor cell as a diffusion medium.

Before each experiment, the mucosal membranes were defrosted at room temperature and mounted between the donor and receptor chambers and clamped using a metal clamp. The receptor chamber that has a magnetic stirrer was filled with the diffusion medium before mounting the membranes and air bubbles were removed after mounting the membranes and before the experiment. The water bath was set at 37° C. for the circulating water in the jacketed Franz Cells. The mounted membranes were equilibrated with the diffusion medium for 30 min from both sides before the experiment and were checked for any leaks.

EpiBit equivalent to 20 mg Epi was dissolved in 2 mL of Mcvilian buffer (n=4) at specific pH including pH 5, 6, 6.8, 7.4, and 8.

Aliquots were withdrawn from the receptor chamber using 6 inch-long needles (Popper &Sons, Inc, New Hyde Park, NY) and 1 mL syringes at 5, 10, 15, 20, 30, 45, 60, 75, and 90 min. The withdrawn volumes were replaced with fresh medium.

Samples were transferred to HPLC vials for HPLC analysis according to USP Method.

The cumulative amount of the Epi permeated over time (AUC₀₋₉₀ₘᵢₙ, μg/cm²/min), influx (J, μg/cm²/min), and permeability coefficient (P, cm/min) were calculated and statistically compared using ANOVA and Tukey-Kramer Tests.

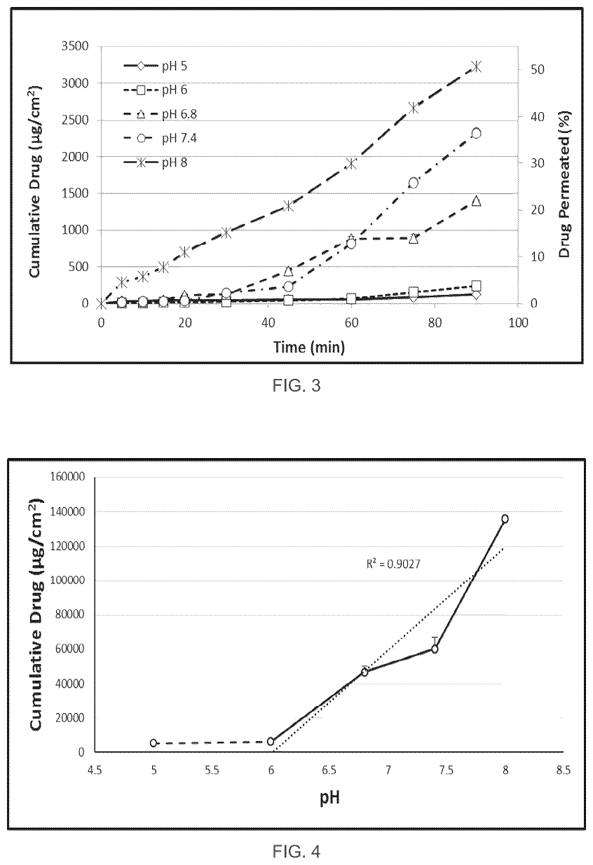

The mean±SD (n=4) cumulative diffused Epi per area at various pH were presented in Table 3 and FIG. 3. The Mean percentage of diffused Epi per area at various pH were presented in Table 4 and FIG. 3.

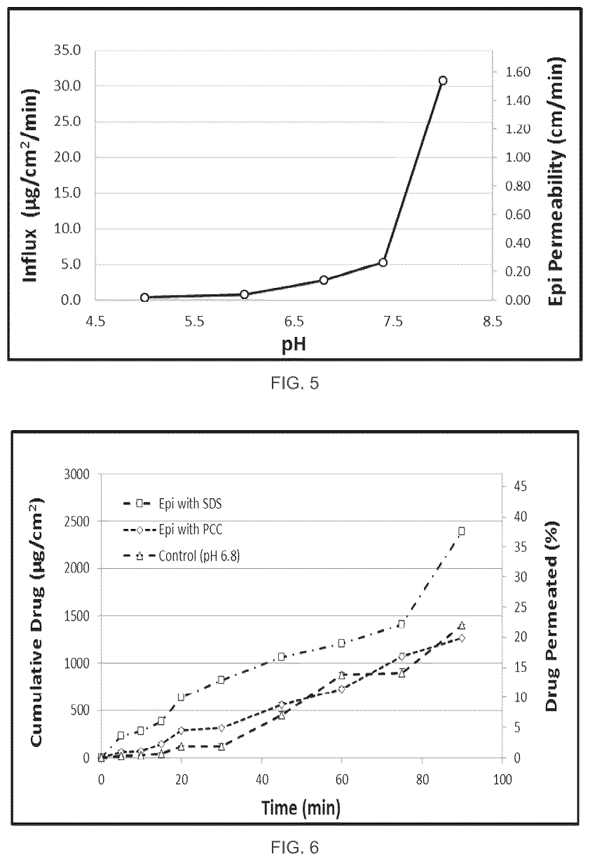

The mean (±SD) Epi AUC₀₋₉₀ₘᵢₙ, J, P were presented in Table 5. Also, Epi AUC₀₋₉₀ₘᵢₙ influx (J) and permeability (P) at each pH were illustrated in FIGS. 4 and 5. AUC₀₋₉₀ J, P at the various pH were significantly different (p<0.05) from each other except at pH 5 and 6, which were not significantly different from each other but. Epi AUC₀₋₉₀ₘᵢₙ increased linearly (R²=0.9) as the pH was increase from 6 to 8. At pH 8 Epi sublingual permeability was increased about 11-fold compared to at pH 6.8, which is the average pH of human saliva.

pH had a significant effect on Epi sublingual permeability. As the pH was increased, the degree of ionization was decreased and result in increasing the sublingual permeation of Epi.

Increasing the pH of the diffusion medium and the subsequent reduction in Epi ionization can result in a significant increase in the Epi sublingual permeation.

TABLE 3
Mean ± SD (n = 4) cumulative permeated epinephrine (20 mg) per area
(μg/cm²) through porcine sublingual mucosa at various pH.

| Time (min) | pH 5 | pH 6 | pH 6.6 | pH 7.4 | pH 8 |
|---|---|---|---|---|---|
| 5 | 33.8 ± 1.8 | 4.7 ± 1.5 | 20.2 ± 1.2 | 18.1 ± 0.7 | 283.6 ± 1.7 |
| 10 | 37.5 ± 0.6 | 7.7 ± 1.6 | 27.6 ± 0.4 | 22.2 ± 1.3 | 367.9 ± 3.0 |
| 15 | 40.3 ± 1.3 | 11.6 ± 1.0 | 44.9 ± 3.0 | 29.2 ± 1.1 | 491.3 ± 9.3 |
| 20 | 43.6 ± 1.1 | 15.7 ± 3.3 | 115.3 ± 2.6 | 33.1 ± 0.6 | 700.0 ± 7.0 |
| 30 | 46.7 ± 1.4 | 23.2 ± 6.0 | 119.9 ± 2.9 | 145.8 ± 3.8 | 967.0 ± 14.4 |
| 45 | 50.7 ± 1.5 | 43.3 ± 15.5 | 447.5 ± 212.7 | 232.4 ± 6.6 | 1325.7 ± 11.7 |
| 60 | 58.7 ± 3.9 | 66.4 ± 3.6 | 877.6 ± 24 | 810.2 ± 156.9 | 1902.2 ± 19.1 |
| 75 | 78.2 ± 7.5 | 147.8 ± 41.1 | 889.7 ± 3.2 | 1644.8 ± 317.7 | 2667.9 ± 30.7 |
| 90 | 118.1 ± 3.5 | 240.0 ± 68.5 | 1401.5 ± 85.0 | 2316.0 ± 4.6 | 3226.6 ± 29.9 |

TABLEs 4
Mean ± SD (n = 4) percentage of diffused epinephrine
(%) through porcine sublingual mucosa at various pH.

| Time (min) | pH 5 | pH 6 | pH 6.8 | pH 7.4 | pH 8 |
|---|---|---|---|---|---|
| 5 | 0.5 ± 0.03 | 0.1 ± 0.02 | 0.3 ± 0.02 | 0.3 ± 0.01 | 4.5 ± 0.03 |
| 10 | 0.6 ± 0.01 | 0.1 ± 0.02 | 0.4 ± 0.01 | 0.3 ± 0.02 | 5.8 ± 0.05 |
| 15 | 0.6 ± 0.02 | 0.2 ± 0.02 | 0.7 ± 0.05 | 0.5 ± 0.02 | 7.7 ± 0.15 |
| 20 | 0.7 ± 0.02 | 0.2 ± 0.05 | 1.8 ± 0.04 | 0.5 ± 0.01 | 11.0 ± 0.11 |
| 30 | 0.7 ± 0.02 | 0.4 ± 0.09 | 1.9 ± 0.05 | 2.3 ± 0.06 | 15.2 ± 0.23 |
| 45 | 0.8 ± 0.02 | 0.7 ± 0.24 | 7.0 ± 3.34 | 3.6 ± 0.10 | 20.8 ± 0 18 |
| 60 | 0.3 ± 0.06 | 1.0 ± 0.06 | 13.8 ± 0.04 | 12.7 ± 2.46 | 29.9 ± 0.30 |
| 75 | 1.2 ± 0.12 | 2.3 ± 0.64 | 14.0 ± 0.05 | 25.8 ± 1.99 | 41.9 ± 0.48 |
| 90 | 1.9 ± 0.6 | 3.8 ± 1.08 | 22.0 ± 1.33 | 36.4 ± 0.07 | 50.7 ± 0.47 |

TABLE 5
Mean ± SD (n = 4) Epi AUC_{0-90 min}, J, P through porcine sublingual mucosa at various pH.

| Time (min) | pH 5 | pH 6 | pH 6.8 | pH 7.4 | pH 8 |
|---|---|---|---|---|---|
| AUC_{0-90 min} | 5169 ± 255* | 6191 ± 756* | 46562 ± 3450 | 60099 ± 6576 | 135689 ± 439 |
| (μg/cm²/ min) | | | | | |
| J (μg/cm²/min) | 0.4 ± 0.1* | 0.8 ± 0.2* | 2.8 ± 0.2 | 5.3 ± 0.2 | 30.9 ± 0.4 |
| P (cm/min) | 0.02 ± 0.00* | 0.04 ± 0.01* | 0.14 ± 0.01 | 0.26 ± 0.01 | 1.54 ± 0.02 |

*p < 0.05 from pH 6.8, 7.4, and 8.

### Example 3: Altering the Microenvironment pH of the Absorption Medium Using pH-Modifying Excipients

Alkalizing (pH-modifying) excipients were selected and evaluated based on the desired pH values that resulted in optimal Epi stability and permeability from previous Examples 1 and 2.

Three pH modifiers were selected, sodium bicarbonate, calcium citrate, and sodium carbonate, and used in various percentages to determine in vitro the amount needed to modify the pH of a 2 mL water as a diffusion medium (n=3) with or without adding EpiBit equivalent to 20 mg Epi. The mean±SD of the medium pH was calculated.

The mean (±SD) of pH readings using various pH-modifying excipients with and without EpiBit equivalent to 20 mg Epi are presented in Table 6. Na Carbonate resulted in the highest mean (±SD) pH with the lowest needed percentage (0.75% w/v) with and without Epi, 8.6±0.2 and 11.7±0.0.

TABLE 6
Mean ± SD (n = 3) of pH measurements using various pH-
modifying excipients with and without epinephrine (20 mg).

| | pH Reading* | | | | | |
|---|---|---|---|---|---|---|
| | Na Bicarbonate | | Na Citrate | | Na Carbonate | |
| | Without | With | Without | With | Without | With |
| Concentration** | Epi | Epi | Epi | Epi | Epi | Epi |
| 0.075% | 8.6 ± 0.06 | — | 7.4 ± 0.1 | — | — | — |
| 0.15% | 8.7 ± 0 06 | 4.5 ± 0.2 | 8.0 ± 0.3 | 3.7 ± 0.1 | 10 ± 0.0 | 4.7 ± 0.3 |
| 0.375% | 8.8 ± 0.06 | 4.9 ± 0.2 | 8.6 ± 0.1 | 4.1 ± 0.1 | 11.7 ± 0.1 | 6.8 ± 0.1 |
| 0.75% | 8.9 ± 0.0 | 6.9 ± 0.0 | 9.3 ± 0.1 | 4.8 ± 0.0 | 11.7 ± 0.0 | 8.6 ± 0.2 |
| 1.125% | 8.9 ± 0.1 | 7.9 ± 0.1 | 9.4 ± 0.1 | 5.0 ± 0.1 | | |
| 1.5% | 8.4 ± 0.1 | 7.3 ± 0.1 | 9.8 ± 0.0 | 6.6 ± 0.2 | | |
| 1.875% | 8.5 ± 0.1 | 7.7 ± 0.0 | 9.8 ± 0.1 | 7.1 ± 0.1 | | |
| 2.25% | 8.6 ± 0.1 | 7.9 ± 0.1 | — | — | | |

*Results presented as mean ± SD pH measurement in 2 mL water, equivalent to average saliva volume in 2 min.
**Percentage (w/v) was calculated for 2 mL water and EpiBit equivalent to 20 mg Epi.

### Example 4: Effect of Penetration (Permeation) Enhancers on Epinephrine Sublingual Permeability

The purpose was to evaluate the effect of different permeability enhancers on the sublingual permeability of Epi. Various penetration enhancers at different concentrations have been investigated to enhance drug permeation through buccal and sublingual routes (20). The effect of sodium dodecyl sulfate (SDS) and palmitoyl carnitine chloride (PCC), as penetration enhancers, on Epi sublingual permeability were evaluated. EpiBit (n=4) equivalent to 20 mg Epi and SDS (0.075% w/v) or PCC (1.2% w/v) were dissolved in 2 mL of phosphate buffer at pH 6.8 (average saliva pH). Epi permeability with no penetration enhancer was used as a control. Excised porcine sublingual membrane were used, as previously described in Example 2, to evaluate the permeation of Epi through static vertical Franz diffusion cells at a controlled temperature of 37° C., as previously described in Example 2. EpiBit solution with or without a penetration enhancer was added to the donor chamber and phosphate buffer at pH 7.4 (pH of the blood) was used in the receiver chamber. Aliquots, 200 μL, were withdrawn from the receiver chamber at several time intervals. The volumes withdrawn were replenished with fresh phosphate buffer and the collected samples were filtered and transferred into HPLC vials for HPLC analysis using a UV detector. The cumulative amount of the Epi permeated over time (AUC₀₋₉₀ₘᵢₙ, μg/cm²/min), influx (J, μg/cm²/min), and permeability coefficient (P, cm/min) were calculated and statistically compared using ANOVA and Tukey-Kramer Tests.

The mean±SD (n=4) cumulative diffused Epi per area with different permeation enhancers are presented in Table 8 and FIG. 6. The Mean percentage of diffused Epi per area with different permeation enhancers were presented in Table 9 and FIG. 6.

TABLE 8
Mean ± SD (n = 4) cumulative permeated
epinephrine (20 mg) per area (μg/cm²) through
porcine sublingual mucosa with various permeation enhancers.

| Time (min) | Control | Epi with SDS | Epi with PCC |
|---|---|---|---|
| 5 | 20.2 ± 1.2 | 228.0 ± 57.4 | 57.1 ± 9.3 |
| 10 | 27.9 ± 0.4 | 280.1 ± 122.3 | 71.9 ± 7.1 |
| 15 | 44.9 ± 3.0 | 380.8 ± 84.0 | 141.0 ± 12.9 |
| 20 | 115.3 ± 2.6 | 631.4 ± 116.0 | 285.7 ± 20.2 |
| 30 | 119.9 ± 2.9 | 817.7 ± 120.7 | 312.4 ± 17.2 |
| 45 | 447.5 ± 212.7 | 1059.2 ± 181.7 | 553.4 ± 27.2 |
| 60 | 877.6 ± 2.4 | 1202.1 ± 259. 7 | 726.7 ± 18.7 |
| 75 | 889.7 ± 3.2 | 1408.9 ± 339.9 | 1070.3 ± 31.7 |
| 90 | 1401.5 ± 85.0 | 2388.3 ± 327.3 | 1262.9 ± 17.7 |

TABLE 9
Mean ± SD (n = 4) percentage of diffused epinephrine (%) through
porcine sublingual mucosa with various permeation enhancers.

| Time (min) | Control | Epi with SDS | Epi with PCC |
|---|---|---|---|
| 5 | 0.3 ± 0.02 | 3.6 ± 0.9 | 0.9 ± 0.1 |
| 10 | 0.4 ± 0.01 | 4.4 ± 1.9 | 1.1 ± 0.1 |
| 15 | 0.7 ± 0.05 | 6.0 ± 1.3 | 2.2 ± 0.2 |
| 20 | 1.8 ± 0.04 | 9.9 ± 1.8 | 4.5 ± 0.3 |
| 30 | 1.9 ± 0.05 | 12.8 ± 1.9 | 4.9 ± 0.3 |
| 45 | 7.0 ± 3.34 | 16.6 ± 2.9 | 8.7 ± 0.4 |
| 60 | 13.8 ± 0.04 | 18.9 ± 4.1 | 11.4 ± 0.3 |
| 75 | 14.0 ± 0.05 | 22.1 ± 5.3 | 16.8 ± 0.5 |
| 90 | 22.0 ± 1.33 | 37.5 ± 5.1 | 19.8 ± 0.3 |

Mean (±SD) AUC₀₋₉₀ₘᵢₙ, J, and P of permeated Epi with SDS 0.075% and PCC 0.75% were statistically higher (p<0.05) than the control. The AUC₀₋₉₀ₘᵢₙ, J, and P of permeated Epi with SDS enhancer were significantly higher (p<0.05) than with PCC enhancer (Table 10). Adding SDS 0.075% achieved the highest enhancement in Epi sublingual permeability and increased Epi permeability about 10-fold compared to control.

TABLE 10
Mean ± SD (n = 4) Epi AUC₀₋₉₀, J, P through porcine
sublingual mucosa with various permeation enhancers.

| | Control | Epi with SDS | Epi With PCC |
|---|---|---|---|
| AUC_{0-90 min} | 46562 ± 3450* | 92365 ± 16594* | 52127 ± 670* |
| (μg/cm²/min) | | | |
| J (μg/cm²/min) | 2.8 ± 0.2* | 29 ± 4.3* | 8.7 ± 0.8* |
| P (/cm/min) | 0.14 ± 0.0* | 1.4 ± 0.2* | 0.4 ± 0.0* |

*p < 0.05 from all.

Penetration enhancers were able to enhance the sublingual permeability of Epi. SDS was superior to PCC in enhancing the sublingual permeability of Epi. The incorporation of a permeability enhancer into Epi formulation can significantly enhance the permeability and potentially the sublingual absorption of Epi as an alternative route of administration for the treatment of anaphylaxis and other conditions responsive to Epi.

### Example 5: Combined Effect of a Penetration (Permeation) Enhancer and pH-Modification on Epinephrine Sublingual Permeability

The combined effect of the alkalizing agent that was able to generate a pH value that resulted in optimal Epi sublingual permeability from previous Examples 2 and 3 with the penetration enhancer that resulted in optimal Epi sublingual permeability from previous Example 4 on the overall sublingual Epi permeability was evaluated. EpiBit equivalent to 20 mg Epi, 0.75% w/v sodium carbonate (Na Carb), as an alkalizing agent, and 0.075% w/v SDS, as a penetration enhancer, were dissolved in 2 mL of deionized water based on results obtained from previous examples. Epi permeability from phosphate buffer at pH 6.8 with no penetration enhancer and no alkalizing agent from previous examples was used as a control. Excised porcine sublingual membranes were used, as described previously in Example 2, to evaluate the permeation of Epi through static vertical Franz diffusion cells at a controlled temperature of 37° C., as described previously in Example 2. EpiBit solution with penetration enhancer and alkalizing agent was added to the donor chamber. Phosphate buffer at pH 7.4 (pH of the blood) was used in the receiver chamber. Aliquots, 200 μL, were withdrawn from the receiver chamber at several time intervals. The volumes withdrawn were replenished with fresh phosphate buffer and the collected samples were filtered and transferred into HPLC vials for HPLC analysis using a UV detector. The cumulative amount of the Epi permeated over time (AUC₀₋₉₀ₘᵢₙ, μg/cm²/min), influx (J, μg/cm²/min), and permeability coefficient (P, cm/min) were calculated and statistically compared with data of Epi permeability at pH 8 from Example 2, Epi permeability with 0.075% w/v SDS alone from Example 4, and control using ANOVA and Tukey-Kramer Tests.

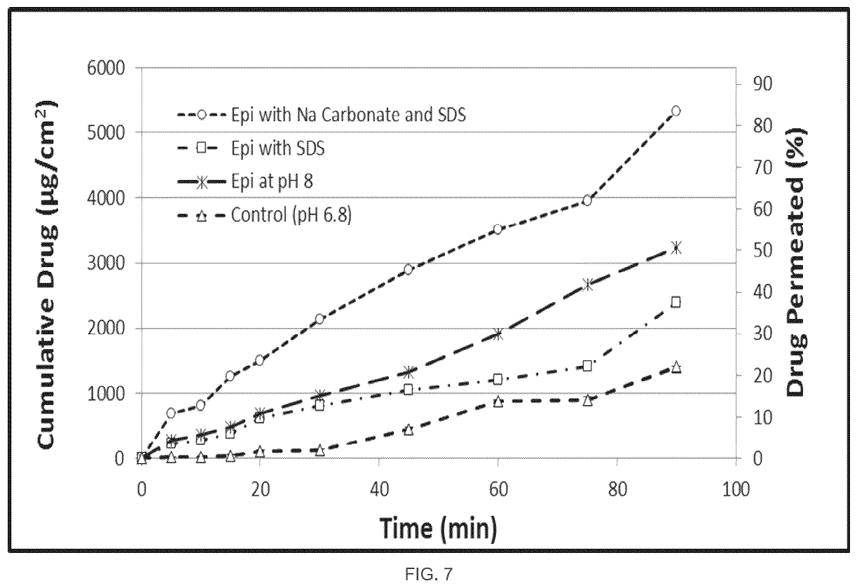

The mean±SD (n=4) cumulative diffused Epi per area with alkalizing agent and permeation enhancer in comparison to Epi at pH 8, Epi with SDS only and control are presented in Table 11 and FIG. 7. The Mean percentage of diffused Epi per area with alkalizing agent and permeation enhancer in comparison to Epi at pH 8, Epi with SDS only, and control were presented in Table 12 and FIG. 7.

TABLE 11
Mean ± SD (n = 4) cumulative permeated epinephrine (20 mg)
per area (μg/cm²) through porcine sublingual mucosa from various formulations.

| | | | | Epi with Na |
|---|---|---|---|---|
| | | | | Carbonate |
| Time (min) | Control | Epi at pH 8 | Ept with SDS | and SDS |
| 5 | 20.2 ± 1.2 | 283.6 ± 1.7 | 228.0 ± 57.4 | 694.8 ± 72.5 |
| 10 | 27.9 ± 0.4 | 367.9 ± 3.0 | 280.1 ± 122.3 | 809.2 ± 51.1 |
| 15 | 44.9 ± 3.0 | 491.3 ± 9.3 | 380.8 ± 84.0 | 1263.2 ± 82.5 |
| 20 | 115.3 ± 2.6 | 700.0 ± 7.0 | 631.4 ± 116.0 | 1506.5 ± 32.4 |
| 30 | 119.9 ± 2.9 | 967.0 ± 14.4 | 817.7 ± 120.7 | 2139.8 ± 150.9 |
| 45 | 447.5 ± 212.7 | 1325.7 ± 11.7 | 1059.2 ± 181.7 | 2888.7 ± 448.4 |
| 60 | 877.6 ± 2.4 | 1902.2 ± 19.1 | 1202.1 ± 259.7 | 3515.9 ± 347.5 |
| 75 | 889.7 ± 3.2 | 2667.9 ± 30.7 | 1408.9 ± 339.9 | 3948.3 ± 139.2 |
| 90 | 1401.5 ± 85.0 | 3226.6 ± 29.9 | 2388.3 ± 327.3 | 5326.2 ± 47.4 |

TABLE 12
Mean ± SD (n = 4) percentage of diffused epinephrine (%) through
porcine sublingual mucosa with various permeation enhancers.

| | | | | Epi with Na |
|---|---|---|---|---|
| | | | | Carbonate |
| Time (min) | Control | Epi at pH 8 | Epi with SDS | and SDS |
| 5 | 0.3 ± 0.02 | 4.5 ± 0.03 | 3.6 ± 0.9 | 10.9 ± 1.1 |
| 10 | 0.4 ± 0.01 | 5.8 ± 0.05 | 4.4 ± 1.9 | 12.7 ± 0.8 |
| 15 | 0.7 ± 0.05 | 7.7 ± 0.15 | 6.0 ± 1.3 | 19.8 ± 1.3 |
| 20 | 1.8 ± 0.04 | 11.0 ± 0.11 | 9.9 ± 1.8 | 23.7 ± 0.5 |
| 30 | 1.9 ± 0.05 | 15.2 ± 0.23 | 12.8 ± 1.9 | 33.6 ± 2.4 |
| 45 | 7.0 ± 3.34 | 20.8 ± 0.18 | 16.6 ± 2.9 | 45.4 ± 7.0 |
| 60 | 13.8 ± 0.04 | 29.9 ± 0.30 | 18.9 ± 4.1 | 55.2 ± 5.5 |
| 75 | 14.0 ± 0.05 | 41.9 ± 0.48 | 22.1 ± 5.3 | 62 0 ± 2.2 |
| 90 | 22.0 ± 1.33 | 50.7 ± 0.47 | 37.5 ± 5.1 | 83.6 ± 0.7 |

Mean (±SD) AUC₀₋₉₀ₘᵢₙ, J, and P of permeated Epi with Na Carbonate (0.75% w/v) and SDS (0.075% w/v) were statistically higher (p<0.05) than all. The J and P of permeated Epi with SDS enhancer alone and at pit H alone were significantly higher (p<0.05) than control but were not significantly different (p>0.05) from each other (Table 13). The combine effect of adding Na Carbonate (0.75% w/v) with SDS 0.075% on Epi sublingual permeability enhancement was significantly higher than using each one alone and increased Epi permeability about 25-fold compared to control.

TABLE 13
Mean ± SD (n = 4) Epi AUC_{0-90 min}, J, P through
porcine sublingual mucosa from various formulations.

| | | | | Epi with Na |
|---|---|---|---|---|
| | | | | Carbonate |
| | Control | Epi at pH 8 | Epi with SDS | and SDS |
| AUC_{0-90 min} | 46562 ± 3450* | 135689 ± 439* | 92365 ± 16594* | 247123 ± 11900* |
| (μg/cm²/min) | | | | |
| J (μg/cm²/min) | 2.8 ± 0.2* | 30.9 ± 0.4** | 29 ± 4.3** | 71.6 ± 0.6* |
| P (/cm/min) | 0.14 ± 0.0* | 1.54 ± 0.0** | 1.4 ± 0.2** | 3.6 ± 0.0* |

*p < 0.05 from all.
**p < 0.05 from control and Epi with Na Carbonate and SDS.

### Example 6: Formulation of Epinephrine Sublingual Composition

A pediatric dose of epinephrine 4 mg and an adult dose of epinephrine 20 mg-40 mg, using epinephrine bitartrate salt, can be formulated as micronized or un-micronized particles with a particle size distribution ranging between 500 nm to 2.5 μm into a sublingual tablet formulation with or without a pH-modifier (alkalizer), with or without a penetration enhancer, with or without a flavor, and/or with or without a taste masking agent.

Example 6a: Formulation of Epinephrine Sublingual Table (15 mg) including 27.29 mg of epinephrine bitartrate sale, a pH modifier, a penetration enhancer, a flavor, and a taste masking agent

| | | Percentage |
|---|---|---|
| Ingredient | Type | (%) |
| active ingredient | Epinephrine Bitartrate | 24.26 |
| fillers | Ceolus, MCC (PH-F20JP) | 6.11 |
| fillers | Ceolus, MCC (UF-702) | 36.66 |
| flavor/sweetener | Mannitol | 14.5 |
| flavor | Grape | 0.5 |
| disintegrant | L-HPC (LH-NBD) (L-HPC:MCC, 1:9) | 5.89 |
| taste masking agent | Citric Acid (CA:API, 1:29) | 0.84 |
| penetration enhancer | SDS (1% of API) | 0.24 |
| akalizer | Na Carb | 10.0 |
| lubricant | Mg Stearate | 1.0 |
| Tablet Weight | | 100.0 |

Example 6b: Formulation of Epinephrine Sublingual Table (15 mg) including 27.29 mg of epinephrine bitartrate sale, a pH modifier, a penetration enhancer, a flavor, and a taste masking agent

| | | Percentage |
|---|---|---|
| Ingredient | Type | (%) |
| active ingredient | Epinephrine Bitartrate | 18.19 |
| fillers | Ceolus, MCC (PH-F20JP) | 6.93 |
| fillers | Ceolus, MCC (UF-702) | 41.55 |
| flavor/sweetener | Mannitol | 14.5 |
| flavor | Grape | 0.5 |
| disintegrant | L-HPC (LH-NBD) (L-HPC:MCC, 1:9) | 6.52 |
| taste masking agent | Citric Acid (CA:API, 1:29) | 0.63 |
| penetration enhancer | SDS (1% of API) | 0.18 |
| akalizer | Na Carb | 10.0 |
| lubricant | Mg Stearate | 1.0 |
| Tablet Weight | | 100.0 |

Example 6c: Formulation of Epinephrine Sublingual Table (15 mg) including 27.29 mg of epinephrine bitartrate sale, a pH modifier, a penetration enhancer, a flavor, and a taste masking agent

| | | Percentage |
|---|---|---|
| Ingredient | Type | (%) |
| active ingredient | Epinephrine Bitartrate | 24.26 |
| fillers | Ceolus, MCC (PH-F20JP) | 6.22 |
| fillers | Ceolus, MCC (UF-702) | 37.31 |
| flavor/sweetener | Mannitol | 14.5 |
| flavor | Grape | 0.5 |
| disintegrant | L-HPC (LH-NBD) (L-HPC:MCC, 1:9) | 5.97 |
| taste masking agent | | |
| penetration enhancer | SDS (1% of API) | 0.24 |
| akalizer | Na Carb | 10.0 |
| lubricant | Mg Stearate | 1.0 |
| Tablet Weight | | 100.0 |

Example 6d: Formulation of Epinephrine Sublingual Table (15 mg) including 27.29 mg of epinephrine bitartrate sale, a pH modifier, a penetration enhancer, a flavor, and a taste masking agent

| | | Percentage |
|---|---|---|
| Ingredient | Type | (%) |
| active ingredient | Epinephrine Bitartrate | 18.19 |
| fillers | Ceolus, MCC (PH-F20JP) | 7.01 |
| fillers | Ceolus, MCC (UF-702) | 42.0 |
| flavor/sweetener | Mannitol | 14.5 |
| flavor | Grape | 0.5 |
| disintegrant | L-HPC (LH-NBD) (L-HPC:MCC, 1:9) | 6.58 |
| taste masking agent | | |
| penetration enhancer | SDS (1% of API) | 0.18 |
| akalizer | Na Carb | 10.0 |
| lubricant | Mg Stearate | 1.0 |
| Tablet Weight | | 100.0 |

Example 6e: Formulation of Epinephrine Sublingual Table (15 mg) including 27.29 mg of epinephrine bitartrate sale, a pH modifier, a penetration enhancer, a flavor, and a taste masking agent

| | | Percentage |
|---|---|---|
| Ingredient | Type | (%) |
| active ingredient | Epinephrine Bitartrate | 16.54 |
| fillers | Ceolus, MCC (PH-F20JP) | 7.15 |
| fillers | Ceolus, MCC (UF-702) | 42.89 |
| flavor/sweetener | Mannitol | 15.0 |
| flavor | | |
| disintegrant | L-HPC (LH-NBD) (L-HPC:MCC, 1:9) | 6.69 |
| taste masking agent | Citric Acid (CA:API, 1:29) | 0.57 |
| penetration enhancer | SDS (1% of API) | 0.2 |
| akalizer | Na Carb | 10.0 |
| lubricant | Mg Stearate | 1.0 |
| Tablet Weight | | 100.0 |

### Summary of Examples

Epinephrine is suitable for sublingual administration and would have minimal or no significant degradation due to human saliva's pH and composition. Also, incorporating pH-modifying excipients into the tablet formulation would not compromise Epi stability (except between pH 4 to 5). They can be useful to adjust the variability in the pH of human saliva due to the consumption of various drinks with wide range of acidity and alkalinity. The incorporation of alkalizing agent to maintain the pH of the diffusion medium around 8 with a permeation enhancer like SDS into Epi sublingual table formulation can enhance the Epi sublingual permeability about 25-fold compared to about 10- to 11-fold increase for each one alone.

### CONCLUSION

Epinephrine is an important medication in health systems worldwide for management of life-threatening allergies, i.e. anaphylaxis, cardiac events, i.e. cardiac arrest, and breathing difficulties, i.e. asthma, bronchial asthma, bronchitis, emphysema, and respiratory infections. The invention described and exemplified herein represents a new non-invasive sublingual drug delivery for epinephrine as a potential alternative, patient-friendly, convenient, and cost-effective dosage form, to the standard epi-autoinjectors.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The compositions, formulations, dosages, epinephrine fine particles, methods, procedures, and techniques described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

### REFERENCES

1. Kemp S F, Lockey R F, Simons F E. Epinephrine: the drug of choice for anaphylaxis. A statement of the World Allergy Organization. Allergy. 2008; 63(8):1061-70.
2. McLean-Tooke A P, Bethune C A, Fay A C, Spickett G P. Adrenaline in the treatment of anaphylaxis: what is the evidence? BMJ. 2003; 327(7427):1332-5.
3. Simons K J, Simons F E. Epinephrine and its use in anaphylaxis: current issues. Curr Opin Allergy Clin Immunol. 2010; 10(4):354-61.
4. Soar J. Pumphrey R, Cant A, Clarke S, Corbett A, Dawson P, et al. Emergency treatment of anaphylactic reactions—guidelines for healthcare providers. Resuscitation. 2008; 77(2):157-69.
5. Simons F E. Epinephrine auto-injectors: first-aid treatment still out of reach for many at risk of anaphylaxis in the community. Ann Aller Asthma Immunol. 2009; 102(5):403-9.
6. Simons F E R. Lack of worldwide availability of epinephrine autoinjectors for outpatients at risk of anaphylaxis. Ann Allergy Asthma Immunol. 2005; 94(5):534-8.
7. Korenblat P, Lundie M J, Dankner R E, Day J H. A retrospective study of epinephrine administration for anaphylaxis: how many doses are needed? Allergy Asthma Proc. 1999; 20(6):383-6.
8. Sicherer S H, Forman J A, Noone S A. Use assessment of self-administered epinephrine among food-allergic children and pediatricians. Pediatrics. 2000; 105(2):359-62.
9. Hoffman B B, Taylor P. Neurotransmission: The Autonomic and Somatic Motor Nervous Systems. In: Hardman J G, Limbird L E, Gilman A G, editors. Goodman & Gilman's The Pharmacological Basis of Therapeutics. 9 ed. New York: McGraw-Hill Companies, Inc.; 2001. p. 115-53.
10. Connors K A, Amidon G L, Stella V J. Chemical Stability of Pharmaceuticals: A Handbook for Pharmacists. 2 ed. New York: Wiley-Interscience Publication; 1986. 438-47 p.
11. Bredenberg S. Duberg M, Lennernas B, Lennernas H. Pettersson A, Westerberg M, et al. In vitro and in vivo evaluation of a new sublingual tablet system for rapid oromucosal absorption using fentanyl citrate as the active substance. Eur J Pharm Sci. 2003; 20(3):327-34.
12. Glover E D, Glover P N, Franzon M, Sullivan C R, Cerullo C C, Howell R M, et al. A comparison of a nicotine sublingual tablet and placebo for smoking cessation. Nicotine Tob Res. 2002; 4(4):441-50.
13. Guez S. Efficacy of desensitization via the sublingual route in mite allergy. Chem Immunol Allergy. 2003; 82:62-76.
14. Saxena P, Salhan S, Sarda N. Sublingual versus vaginal route of misoprostol for cervical ripening prior to surgical termination of first trimester abortions. Eur J Obstet Gynecol Reprod Biol. 2006; 125(1):109-13.
15. Rawas-Qalaji M M, Simons F E, Simons K J. Fast-disintegrating sublingual epinephrine tablets: effect of tablet dimensions on tablet characteristics. Drug Dev Ind Pharm. 2007; 33(5):523-30.
16. Rawas-Qalaji M M. Simons F E R, Simons K J. Fast-Disintegrating Sublingual Tablets: Effect of Epinephrine Load on Tablet Characteristics. AAPS PharmSciTech. 2006; 7(2):E72-E8.
17. Birudaraj R, Berner B, Shen S, Li X. Buccal permeation of buspirone: mechanistic studies on transport pathways. J Pharm Sci. 2005; 94(1):70-8.
18. Diem K, Lentner C. Scientific Tables. 7ed. Baste, Switz.: Ciba-Geigy Limited; 1971.
19. Goswami T, Li X, Jasti B R. Effect of Lipophilicity and Drug Ionization on Permeation Across Porcine Sublingual Mucosa. AAPS PharmSciTech. 2017; 18(1):175-81.
20. Hassan N, Ahad A, Ali M, Ali J. Chemical permeation enhancers for transbuccal drug delivery. Expert Opin Drug Deliv. 2010; 7(1):97-112.
21. Epinephrine: Chemical and Physical Properties Bethesda, MD. USA PubChem, National Center for Biotechnology Information, U.S. National Library of Medicine; 2018 [https://pubchem.ncbi.nlm.nih.gov/compound/5816#section=Chemical-and-Physical-Properties].
22. Rawas-Qalaji M M, Simons F E, Simons K J. Sublingual epinephrine tablets versus intramuscular injection of epinephrine: Dose equivalence for potential treatment of anaphylaxis. J Allergy Clin Immunol. 2006; 117(2):398-403.
23. Attia M A, El-Gibaly I, Shaltout S E, Fetih G N. Transbuccal permeation, anti-inflammatory activity and clinical efficacy of piroxicam formulated in different gels. Int J Pharm. 2004:276(1-2):11-28.
24. Nicolazzo J A, Reed B L, Finnin B C. Assessment of the effects of sodium dodecyl sulfate on the buccal permeability of caffeine and estradiol. J Pharm Sci. 2004; 93(2):431-40.
25. Duizer E, van der Wulp C, Versantvoort C H, Groten J P. Absorption enhancement, structural changes in tight junctions and cytotoxicity caused by palmitoyl carnitine in Caco-2 and IEC-18 cells. J Pharmacol Exp Ther. 1998; 287(1):395-402.
26. Sutton S C, LeCluyse E L, Cammack L, Fix J A. Enhanced bioavailability of cefoxitin using palmitoyl L-carnitine. 1. Enhancer activity in different intestinal regions. Pharm Res. 1992; 9(2):191-4.
27. Swenson E S, Milisen W B, Curatolo W. Intestinal permeability enhancement: efficacy, acute local toxicity, and reversibility. Pharm Res. 1994; 11(8):1132-42.
28. R. H. Müller et al. Eur J Pharm Biopharm 78(1):1-9 2011.

1. A sublingual composition formulated as a fast-disintegrating tablet, the sublingual composition comprising:
epinephrine bitartrate fine particles; and
an alkalizing excipient for increasing pH at a site of administration of the sublingual composition.

2. The sublingual composition according to claim 1, wherein the epinephrine bitartrate fine particles have a particle size distribution in a range from about 500 nm to about 2.5 μm.

3. (canceled)

4. The sublingual composition according to claim 1, wherein the epinephrine bitartrate is a dosage equivalent ranging from about 15 mg epinephrine to about 20 mg epinephrine.

5-8. (canceled)

9. The sublingual composition according to claim 1, wherein the alkalizing excipient is sodium carbonate, sodium bicarbonate, or calcium citrate.

10. The sublingual composition according to claim 9, wherein the alkalizing excipient is sodium carbonate added to the sublingual composition at 0.75% w/v.

11. The sublingual composition according to claim 1, further comprising a penetration enhancer, wherein the penetration enhancer is sodium dodecyl sulfate (SDS) or palmitoyl carnitine chloride (PCC).

12. The sublingual composition according to claim 11, wherein the penetration enhancer is sodium dodecyl sulfate (SDS) added to the sublingual composition at 0.075% w/v.

13. The sublingual composition according to claim 11, wherein the penetration enhancer is palmitoyl carnitine chloride (PCC) added to the sublingual composition at 1.2% w/v.

14. (canceled)

15. A sublingual composition formulated as a fast-disintegrating tablet, the sublingual composition comprising:
epinephrine bitartrate fine particles;
sodium carbonate; and
sodium dodecyl sulfate (SDS).

16. (canceled)

17. The sublingual composition according to claim 15, wherein the epinephrine bitartrate is a dosage equivalent to about 20 mg epinephrine; sodium carbonate is added to the sublingual composition at 0.75% w/v; and sodium dodecyl sulfate (SDS) ji added to the sublingual composition at 0.075% w/v.

18. The sublingual composition according to claim 15, wherein the epinephrine bitartrate is a dosage equivalent to about 15 mg epinephrine; sodium carbonate is added to the sublingual composition at 0.75% w/v; and sodium dodecyl sulfate (SDS) ji added to the sublingual composition at 0.075% w/v.

19. The sublingual composition according to claim 15, further comprising at least one of a filler, a flavor, a sweetener, a disintegrant, a taste masking agent, and a lubricant.

20-33. (canceled)

34. The sublingual composition according to claim 15, wherein the epinephrine bitartrate fine particles have a dosage equivalent ranging from about 20 mg to about 1 mg epinephrine.

35. The sublingual composition according to claim 15, wherein, the sublingual composition, upon sublingual administration to a subject, controls pH of saliva of the subject within a range optimal for absorption at a site of the sublingual administration.

36. The sublingual composition according to claim 35, wherein the range optimal for absorption is a range of about 6.8 to 8.0.

37. The sublingual composition according to claim 35, wherein the control of the pH of the saliva includes increasing the pH of the saliva of the subject.

38. The sublingual composition according to claim 15, wherein, the sublingual composition, upon sublingual administration to a subject, alters ionization of epinephrine at a site of the sublingual administration for increasing permeability of epinephrine.

39. A method for enhancing sublingual bioavailability of epinephrine in a subject in need thereof, the method comprising:
providing the sublingual composition according to claim 34; and
administering the sublingual composition to the subject.

40. A sublingual composition formulated as a fast-disintegrating tablet, the sublingual composition comprising:
epinephrine bitartrate microparticles;
an alkalizing excipient;
a penetration enhancer;
a filler;
a flavor;
a sweetener;
a disintegrant;
a taste masking agent; and
a lubricant;
wherein the epinephrine bitartrate microparticles have a dosage equivalent ranging from about 20 mg to about 1 mg epinephrine;
wherein the alkalizing excipient is sodium carbonate added to the sublingual composition at 0.75% w/v; and
wherein the penetration enhancer is sodium dodecyl sulfate (SDS) added to the sublingual composition at 0.075% w/v.

41. The sublingual composition according to claim 40, wherein the filler is microcrystalline cellulose (MCC), the flavor is grape, the sweetener is mannitol, the disintegrant is low-substituted hydroxypropyl ether of cellulose (L-HPC), the taste masking agent is citric acid, and the lubricant is magnesium stearate.
